(19) United States
(12) Patent Application Publication
(10) Pub. No.: US 2004/0043499 A1
(43) Pub. Date: Mar. 4, 2004

(54) Dissolved carbon measurement

(76) Inventor: Maria Lee-Alvarez, West Chester, OH (US)

Correspondence Address: KIRKPATRICK & LOCKHART LLP, 535 SMITHFIELD STREET, PITTSBURGH, PA, 15222, US

(21) Appl. No.: 10/228,848

(22) Filed: Aug. 27, 2002

(51) Int. Cl. G01N 33/00

(52) U.S. Cl. 436/146; 422/68.1

(57) ABSTRACT

An analytical instrument measures dissolved carbon in a sample liquid. A syringe pump selectively pumps liquids from valve inlets to a first valve outlet. A sparger receives a sample liquid and a base liquid from the first valve outlet and provides purgeable organic carbon POC gasses. A chemical reactor receives the POC gasses and generates carbon dioxide CO₂. A controller coupled to the analytical instrument provides a control actuation sequence to a valve control input and a pump control input. The CO2 couples to an analyzer. The controller generates a first analyzer output that represents a concentration of dissolved POC in the sample liquid.

### FIELD OF THE INVENTION

The present invention relates generally to chemical instruments that measure the carbon content of a sample. More specifically, the invention relates to chemical instruments that can be used to measure one or more components of total carbon dissolved in a liquid sample.

### BACKGROUND OF THE INVENTION

The measurement of carbon content in liquids such as drinking water, treated or untreated wastewater, and ultrapure water for pharmaceutical or clean room applications is a routine way to assess the purity of the liquid sample. Solid or semi-solid specimens such as soils, clays, or sediments can likewise be measured for carbon content using known analyzer accessories.

Known analyzers have a wet chemistry section with multiple complex flow paths. These analyzers require the use of a number of valves in the flow paths that require maintenance. There is a desire to reduce the complexity of the flow paths, reduce the number of valves, and reduce maintenance requirements for a wet chemistry section of a dissolved carbon analysis instrument.

### SUMMARY OF THE INVENTION

Disclosed is an analytical instrument for measuring dissolved carbon in a sample liquid. The instrument comprises a valve having valve inlets that receive the sample liquid, liquid water and a base liquid. The valve has a valve control input and at least a first valve outlet.

The instrument also comprises a syringe pump coupled to the valve. The syringe pump selectively pumps the liquids from the valve inlets to one or more valve outlets. The syringe pump includes a pump control input.

A sparger is included in the instrument. The sparger receives the sample liquid and a quantity of the base liquid from the first valve outlet. The sparger provides a gas flow to remove purgeable organic carbon POC gasses during a first time interval.

A chemical reactor receives the purgeable organic carbon POC gasses. The chemical reactor generates carbon dioxide during the first time interval.

A control interface couples a control actuation sequence to the valve control input and the pump control input. The carbon dioxide is coupled to an analyzer that couples to a controller that generates a first analyzer output. The first analyzer output represents a concentration of dissolved purgeable organic carbon POC in the sample liquid. The controller also provides the control actuation sequence.

These and various other features as well as advantages that characterize the present invention will be apparent upon reading of the following detailed description and review of the associated drawings.

### BRIEF DESCRIPTION OF THE DRAWINGS

FIG. 1 illustrates a PRIOR ART liquid sample carbon analyzer arrangement.

FIG. 2 schematically illustrates details of the PRIOR ART liquid sample carbon analyzer illustrated in FIG. 1.

FIG. 3 illustrates a block diagram of a first embodiment of a liquid sample carbon analytical instrument.

FIG. 4 illustrates a block diagram of a second embodiment of a liquid sample carbon analytical instrument.

FIG. 5 illustrates a timing diagram of control actuations during purgeable organic carbon POC analysis.

FIG. 6 illustrates a timing diagram of control actuations during inorganic carbon IC analysis.

FIG. 7 illustrates a timing diagram of control actuations during non-purgeable organic carbon NPOC analysis.

### DETAILED DESCRIPTION OF ILLUSTRATIVE EMBODIMENTS

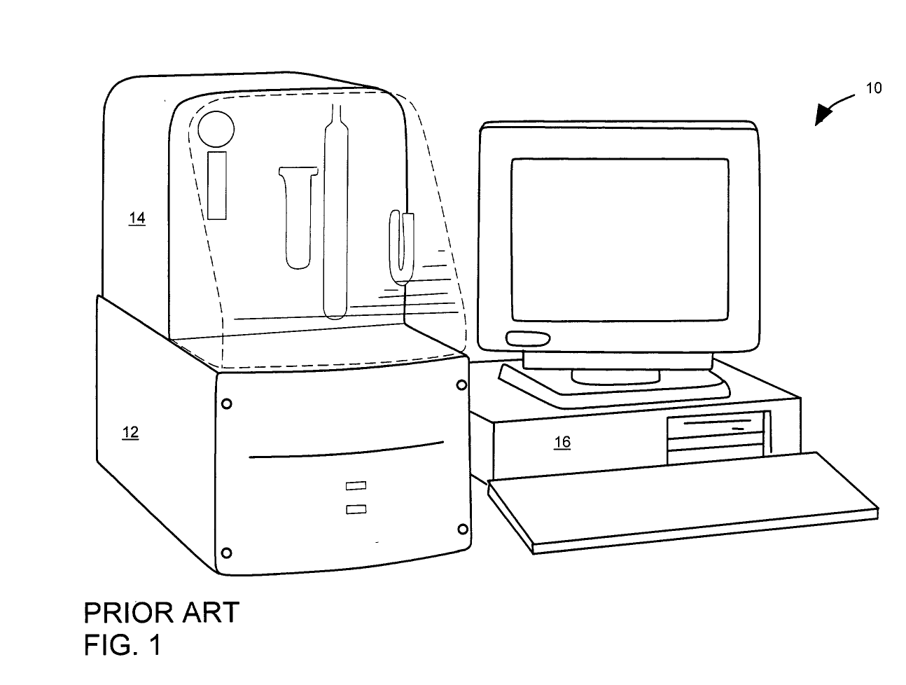

FIG. 1 illustrates a PRIOR ART liquid sample carbon analyzer arrangement 10. The arrangement 10 includes a non-dispersive infrared NDIR gas analyzer 12, a wet chemistry section 14 and a computer 16. An example of the internal operation of the wet chemistry section 14 can be found in U.S. Pat. No. 6,007,777 Purcell et al., which is hereby incorporated herein by reference.

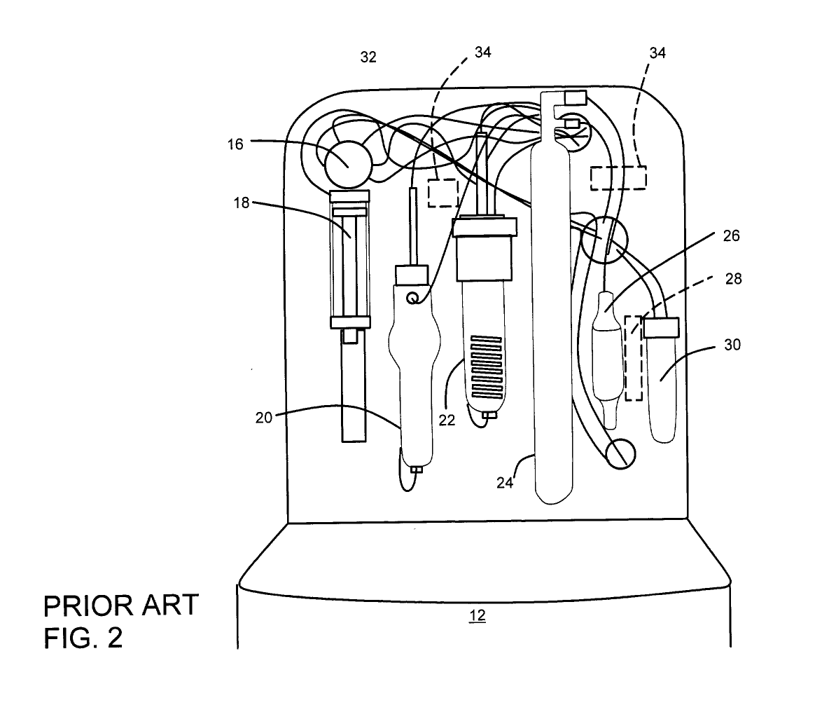

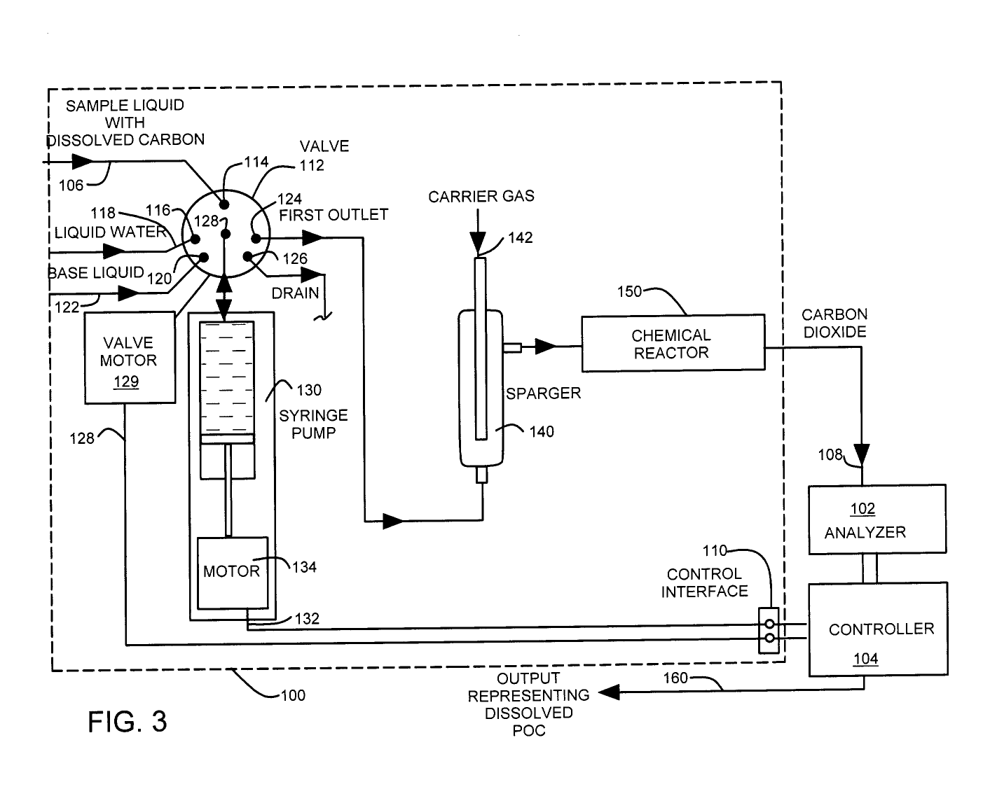

FIG. 2 schematically illustrates details of the wet chemistry section 14 illustrated in FIG. 1. The wet chemistry section 14 includes a valve 16, a syringe pump 18, a sparger 20, a UV reactor 22, a gas-liquid separator 24, a mist trap 26, a nafion tube 28 and a halogen scrubber 30. The components 16, 18, 20, 22, 24, 26, 28, 30 are interconnected by a number of tubes 32 and valves 34 (hidden from view behind mounting plate 36) generally as set forth in U.S. Pat. No. 6,007,777 Purcell et al. The valves and tubes require maintenance. There is a desire to reduce the complexity of the flow path and maintenance requirements for a wet chemistry section. As described below in connection with FIGS. 3-7, a wet chemistry section is provided that has a reduced number of valves and a simplified flow path for carrier gas.

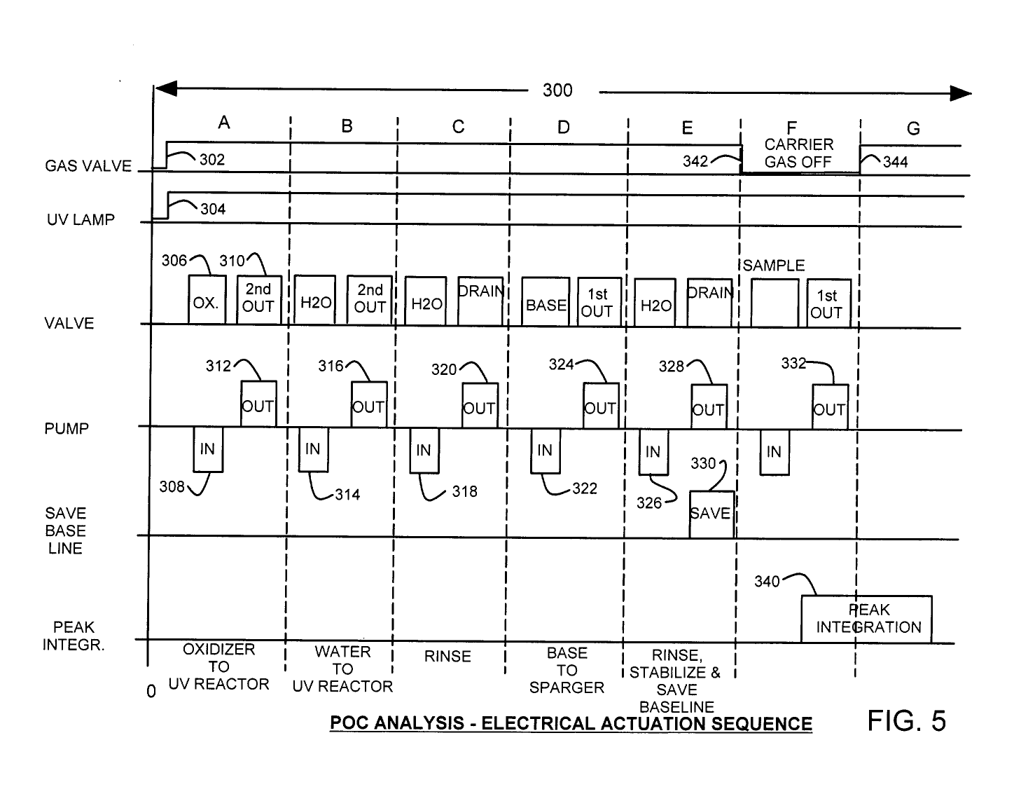

FIG. 3 illustrates a block diagram of a first embodiment of an analytical instrument 100 connected to an analyzer 102. The analytical instrument 100 receives a sample liquid 106 and measures dissolved carbon in the sample liquid 106. Dissolved carbon in the sample liquid 106 is oxidized in a chemical reactor 150 to form carbon dioxide CO₂ gas that is delivered to outlet 108. As the carbon dioxide gas at outlet 108 is produced over a time interval, the carbon dioxide at outlet 108 flows to the analyzer 102 for detection. A signal from analyzer 102 couples to a controller 104. The controller 104 integrates and quantifies the signal from analyzer 102. The controller 104 also couples to a control interface 110 to provide a control actuation sequence to the instrument 100. The control actuation sequence is explained in more detail below in connection with an example shown in FIG. 5.

The instrument 100 includes a valve 112 that has a valve inlet 114 receiving the sample liquid 106, a valve inlet 116 receiving liquid water 118 and a valve inlet 120 receiving a base liquid 122. The liquid water 118 is preferably deionized water, and the base liquid is preferably sodium hydroxide NaOH. The valve 112 also has a first valve outlet 124 and a drain outlet 126. The valve has a primary valve port 128 which the valve 112 can connect to a selected one of the other valve ports 114, 116, 120, 124, 126. The valve 112 includes a valve control input 128 that connects to a valve positioning motor 129 that can be actuated to select one of the valve ports 114, 116, 120, 124,126 to connect to the primary valve port 128.

A syringe pump 130 couples to the primary valve port 128. The syringe pump 130 selectively pumps a selected one of the liquids from the valve inlets 114, 116, 120 to the first valve outlet 124. The syringe pump 130 includes a pump control input 132 for actuating a syringe pump positioning motor 134. The syringe pump 130 can draw in one or more selected liquids from the inlets 114, 116, 120 and then pump out the drawn in liquids to either the first outlet 124 or the drain 126.

A sparger 140 (also called a sparging tube) receives a quantity of the sample liquid 106 and a quantity of the base liquid 122 from the first valve outlet 124. Carrier gas is provided to the sparger 140 at sparger inlet 142. The base liquid 122 and the inorganic carbon IC in the sample liquid 106 react with one another in the sparger 140 such that inorganic carbon IC is retained by the reaction and purgeable organic carbon POC is released by the sparge gas. The sparger 140 provides the purgeable organic carbon POC gasses during a first time interval to a chemical reactor 150.

The chemical reactor 150 receives the purgeable organic carbon POC gasses. The chemical reactor 150 breaks down the purgeable organic carbon POC gasses to produce carbon dioxide CO₂. The chemical reactor 150 generates carbon dioxide during the first time interval. The chemical reactor 150 can be any known chemical reactor which can break down organics and produce carbon dioxide. For example, the chemical reactor 150 can be a UV reactor, which does not use high temperatures, or can be a combustion reactor, which does use high temperatures.

A control interface 110 couples the control actuation sequence to the valve control input 128 and the pump control input 132. The carbon dioxide flows from the chemical reactor 150 to the analyzer 102. The analyzer 102 couples to the controller 104 that provides the control actuation sequence at control interface 110. The controller 104 generates a first analyzer output 160 that represents a concentration of dissolved purgeable organic carbon POC in the sample liquid 106. Depending on the needs of the application, the various actuation inputs can be electrical, pneumatic, optical or other know types of actuation. The first analyzer output 160 can be a display on a computer screen, data stored in memory, or an electrical output from the controller 104 depending on the needs of the application. The control actuation sequence during the first time interval is explained in more detail below in connection with an example timing diagram illustrated in FIG. 5.

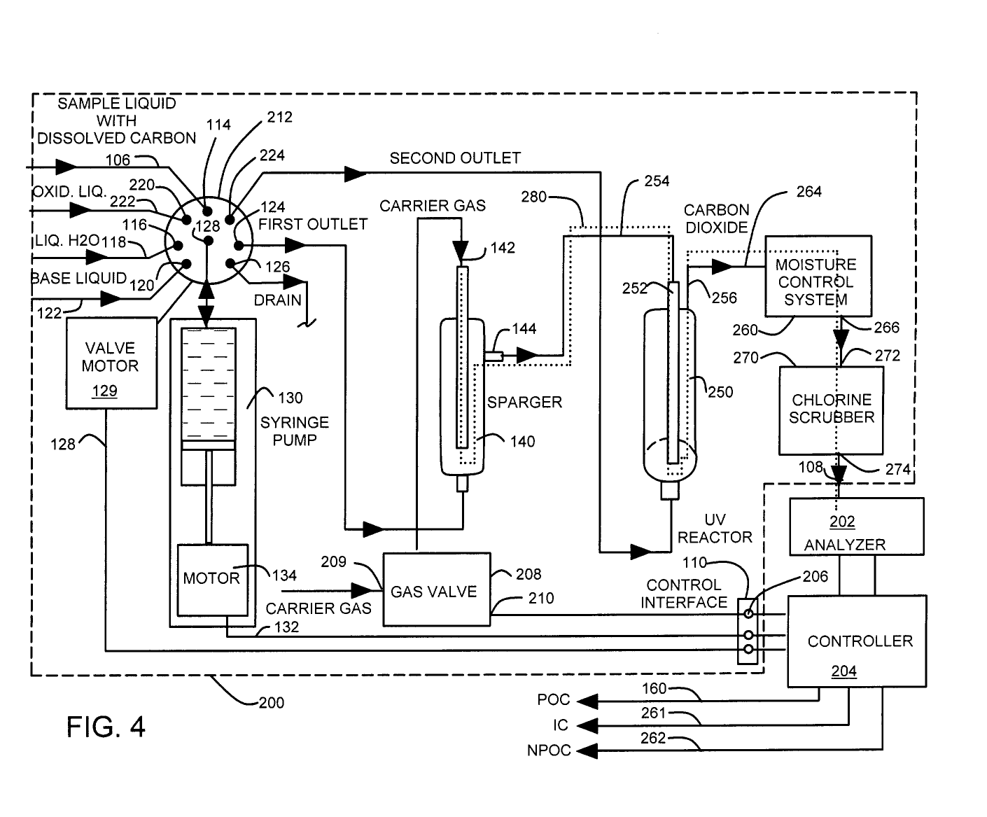

FIG. 4 illustrates a block diagram of a second embodiment of a liquid carbon instrument 200 connected to an analyzer 202. The instrument 200 includes many features that are similar to features in instrument 100. Items in FIG. 4 that have the same reference numerals as items in FIG. 3 are the same or serve the same or a similar function. In addition to perfoming an analysis of POC, the analyzer 200 can also perform an analysis of inorganic carbon IC gasses and non-purgeable organic NPOC. In FIG. 4, a controller 204 coupled to the analyzer 202 includes an output 206 for actuating a gas valve 208 for controlling flow of the carrier gas at sparger inlet 142. Also illustrated in FIG. 4, a UV reactor 250 is coupled to a second outlet 224 on valve 212. The second outlet 224 receives oxidizing liquid 222 from valve inlet 220 and also receives liquid pumped back out of the sparger 140. The oxidizing liquid 222 includes an acid. A moisture control system 260 and a chlorine scrubber 270 are included in the line from the UV reactor 250 to the outlet 108.

The valve 212 includes the valve inlet 220 that receives the oxidizing liquid 222. After the POC analysis is completed, the sparger 140 receives a quantity of the oxidizing liquid 222 and provides inorganic carbon IC gasses to the UV reactor 250 and on to the analyzer 202 during a second time interval. In the UV reactor 250, the UV-promoted oxidation of organics is both physical and chemical in nature. The UV radiation boosts the energy state of molecules and makes them more reactive. In a preferred arrangement, the oxidizing liquid 222 comprises a persulfate, such as sodium, potassium or ammonium persulfate. The persulfate ion in aqueous solution is converted to a sulfate radical by the UV radiation. Hydroxyl radicals are produced by the UV radiation acting on the water molecules and additional hydroxyl radicals are formed by sulfate radicals reacting with the water. The hydroxyl radicals oxidize the carbon in the organic molecules to produce carbon dioxide.

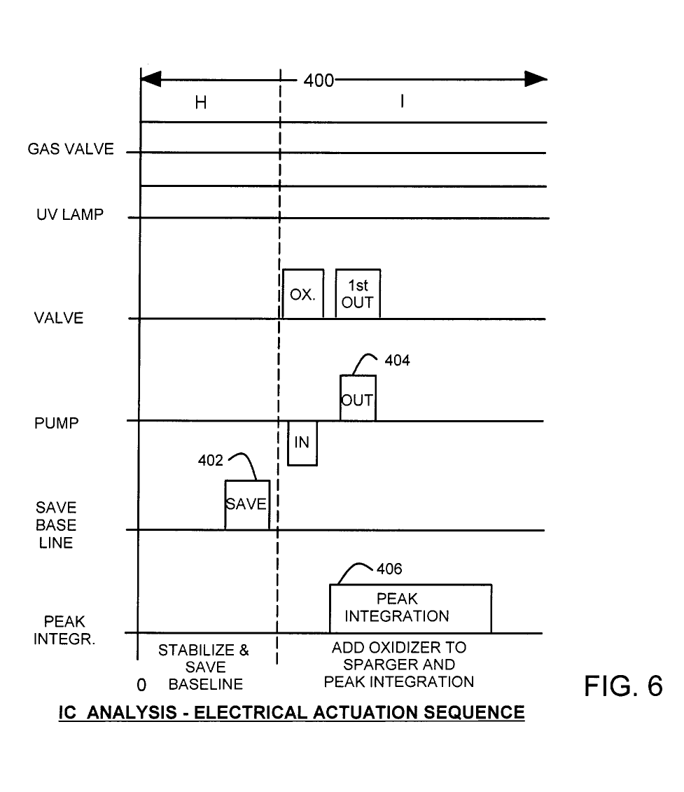

The controller 204 generates a second analyzer output 261 representative of the concentration of dissolved inorganic carbon IC in the sample liquid 106. Actuations during the second time interval are explained in more detail in connection with an example timing diagram illustrated in FIG. 6.

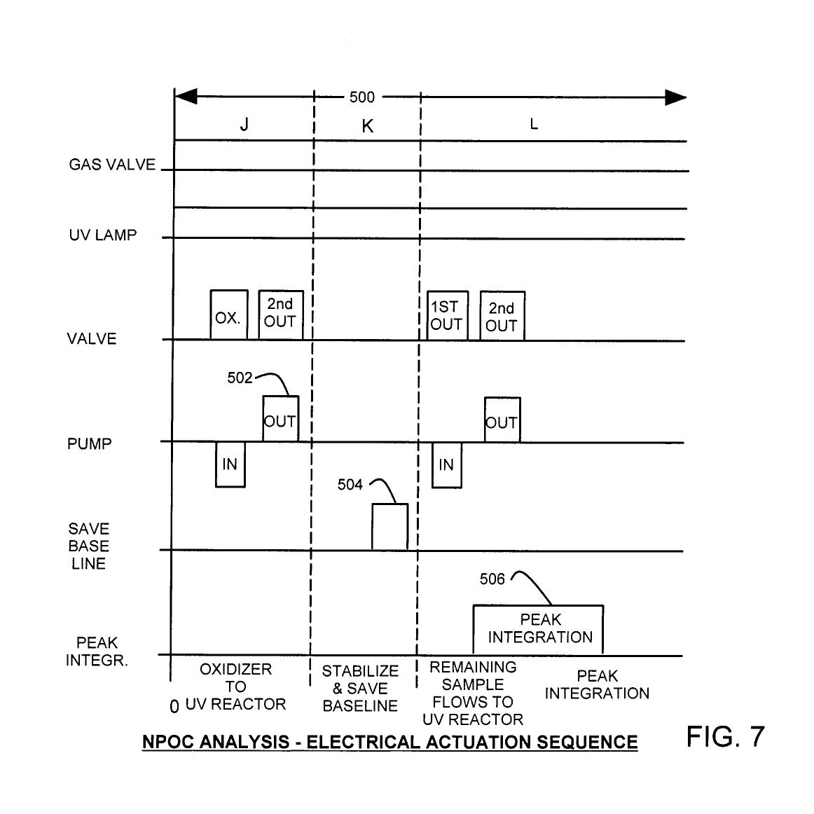

The liquids remaining in the sparger 140 after the second time interval is completed, i.e., during a third time interval, comprise remaining non-purgeable organic carbon NPOC. The liquids remaining in the sparger 140 are provided to the UV chemical reactor 250 during the third time interval. The UV chemical reactor 250 generates carbon dioxide during the third time interval. The controller 204 generates a third analyzer output 262 representative of the concentration of dissolved non-purgeable organic carbon NPOC in the sample liquid 106. Actuations during the third time interval are explained in more detail in connection with an example timing diagram illustrated in FIG. 7.

The sparger 140 comprises a sparger outlet 144 and the UV chemical reactor 250 comprises a chemical reactor inlet 252. A tube 254 connects between the sparger outlet 144 and the chemical reactor inlet 252. The tube 254 forms a portion of a flow path that carries the purgeable organic carbon POC. The carrier gas flows along a flow path 280 from the sparger 140 to the UV chemical reactor 250 to the analyzer 202 and that flow path 280 is uninterrupted by valves.

The gas valve 208 has a gas inlet 209 that receives carrier gas. The gas valve 208 has a gas valve control input 210 that couples to the output 206 of the controller 204. The sparger 140 has a sparger gas inlet 142 that receives the carrier gas from the gas valve 208. The controller 204 provides the control actuation sequence, such as the one described below in connections with FIGS. 5-7 to the gas valve control input 210.

The carrier gas flows along the flow path 280 from the sparger 140 to the UV chemical reactor 250 to the analyzer 202 that is uninterrupted by valves.

The moisture control system MCS 260 has an MCS inlet 264 coupled to a UV reactor outlet 256 to receive the carbon dioxide and has a MCS outlet 266. The scrubber 270 has a scrubber inlet 272 coupled to the MCS outlet 266 and a scrubber outlet 274 providing the carbon dioxide to the analyzer 202.

FIGS. 5-7 illustrate examples of control actuations of various components of the analyzer 200 during successive first, second and third time intervals. POC is analyzed during the first time interval, IC is analyzed during the second time interval, and NPOC is analyzed during the third time interval.

FIG. 5 illustrates a timing diagram during the first time interval 300 that shows control actuations during purgeable organic carbon POC analysis. The timing diagram illustrated in FIG. 5 is described below in connection with the instrument 200 in FIG. 4, however, portions of the timing diagram in FIG. 5 can also be considered in relation to corresponding similar features of the instrument 100 illustrated in FIG. 3.

At the start of the first time interval 300, the gas valve 208 is opened as illustrated at time 302 in FIG. 5 and carrier gas is allowed to flow through the instrument 200 along flow path 280. Also at the start, a UV lamp in the UV reactor 250 is turned on as illustrated at time 304. First time interval 300 includes time phases A, B, C, D, E, F, G. During phase A, the valve 212 is first positioned to connect the syringe pump 130 with the oxidizing liquid inlet 220 as illustrated at time 306 and the syringe pump 130 draws in oxidizing liquid 222 as illustrated at time 308. Next in phase A, the valve 212 is positioned to connect the syringe pump 130 with the second outlet 224 as illustrated at time 310 and the syringe pump 130 pumps out a quantity of the oxidizing liquid 222 to the UV reactor 250 as illustrated at time 312. During phase B, liquid water 118 is drawn in at time 314 and pumped out to the UV chemical reactor 250 as illustrated at time 316. During phase C, the pump is rinsed with water at 318, 320. During phase D base liquid 122 is drawn in at time 322 and pumped out to the sparger 140 at time 324. During phase E, the pump is rinsed with water at times 326, 328 and the controller 204 saves a baseline at time 330. Saving the baseline is essentially a rezeroing of an integrator so that the integrator is ready to integrate a sample. During phase F, the carrier gas is shut off as illustrated at time 342 and a quantity of sample liquid is pumped into the sparger 140 at time 332, and the analyzer 202 begins peak integration of the carbon dioxide at outlet 108 as illustrated at time 340. During phase G, the carrier gas is turned back on at time 344 and peak integration continues to time 346 after substantially all of the POC has reacted in the UV chemical reactor 250. At the end of first time interval 300, the instrument 200, but not the instrument 100, is ready to begin a second time interval explained below in connection with FIG. 6.

FIG. 6 illustrates a timing diagram during a second time interval 400 that shows control actuations during inorganic carbon IC analysis. Second time interval 400 includes time phases H and I. During the phase H, the baseline is saved as illustrated at time 402. During phase I, oxidizer is pumped out to the sparger 140 as illustrated at time 404 and peak integration is performed as illustrated at time 406. Peak integration continues until after substantially all of the CO₂ produced by the inorganic carbon IC is integrated. At the end of second time interval 400, the instrument is ready to begin a third time interval explained below in connection with FIG. 7.

FIG. 7 illustrates a timing diagram of control actuations during a third time interval 500 that shows non-purgeable organic carbon NPOC analysis. Third time interval 500 includes time phases J, K and L. During phase J, additional oxidizer liquid is pumped out to the UV chemical reactor as illustrated at time 502. During phase K, the instrument 200 is allowed to stabilize and a baseline is saved at time 504. During phase L, peak integration is started at time 506 as the remaining sample in the sparger 140 is pumped in by the syringe pump 130 through the first outlet 124 (which acts as an inlet) and pumped out by the syringe pump 130 through the second outlet 224 to the UV chemical reactor 250. Peak integration continues until after substantially all of the CO₂ produced by a reaction of the non-purgeable organic carbon NPOC and the oxidizing liquid is integrated. At the end of the third time interval 500, the analysis of the sample fluid is complete. The controller 204 can calculate a sum of POC+IC+NPOC to provide a measurement of total carbon TC.

It is to be understood that even though numerous characteristics and advantages of various embodiments of the invention have been set forth in the foregoing description, together with details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. For example, the particular elements may vary depending on the particular application for the dissolved carbon analysis while maintaining substantially the same functionality without departing from the scope and spirit of the present invention. The chemical reactor may be a UV persulfate reactor, a combustion reactor or other know type of reactor. In addition, although the preferred embodiment described herein is directed to a benchtop laboratory style of instrument, it will be appreciated by those skilled in the art that an embodiment as a process analyzer can be implemented as well. The teachings of the present invention can be applied to other chemical processing instruments without departing from the scope and spirit of the present invention.

What is claimed is:

1. An analytical instrument for measuring dissolved carbon in a sample liquid, comprising:
a valve having valve inlets receiving the sample liquid, liquid water and a base liquid, the valve having a valve control input, and at least a first valve outlet;
a syringe pump coupled to the valve and selectively pumping the liquids from the valve inlets to the first valve outlet, the syringe pump including a pump control input;
a sparger receiving the sample liquid and a quantity of the base liquid from the first valve outlet, the sparger providing purgeable organic carbon POC gasses during a first time interval;
a chemical reactor receiving the purgeable organic carbon POC gasses, the chemical reactor generating carbon dioxide during the first time interval; and
a control interface adapted to couple a control actuation sequence to the valve control input and the pump control input, and the carbon dioxide being couplable to an analyzer that is coupled to a controller, the controller generating a first analyzer output representing a concentration of dissolved purgeable organic carbon POC in the sample liquid, and generating the control actuation sequence.

2. The analytical instrument of claim 1 wherein the valve includes a valve inlet receiving an oxidizing liquid and the sparger receives a quantity of the oxidizing liquid and provides inorganic carbon IC gasses to the reactor during a second time interval; the reactor generating carbon dioxide during the second time interval and the controller generating a second analyzer output representative of the concentration of dissolved inorganic carbon IC in the sample liquid.

3. The analytical instrument of claim 2 wherein the liquids in the sparger during a third time interval comprise non-purgeable organic carbon NPOC and the liquids are provided to the chemical reactor during the third time interval, and the chemical reactor generates carbon dioxide during the third time interval; the controller generating a third analyzer output representative of the concentration of dissolved non-purgeable organic carbon NPOC in the sample liquid.

4. The analytical instrument of claim 2 wherein the valve further comprises a second valve outlet, and the chemical reactor comprises a UV reactor having a UV reactor liquid input receiving a quantity of the oxidizing liquid from the second valve outlet.

5. The analytical instrument of claim 4, further comprising a gas valve having a gas inlet receiving a carrier gas and a gas valve control input; the sparger having a sparger gas inlet receiving the carrier gas from the gas valve, and the controller providing the control actuation sequence to the gas valve control input.

6. The analytical instrument of claim 5 wherein the carrier gas flows along a flow path from the sparger to the chemical reactor to the analyzer and the flow path is uninterrupted by valves.

7. The analytical instrument of claim 1, further comprising:
a moisture control system MCS having an MCS inlet coupled to the UV reactor outlet to receive the carbon dioxide and a MCS outlet; and
a scrubber having a scrubber inlet coupled to the MCS outlet and a scrubber outlet providing the carbon dioxide to the analyzer.

8. The analytical instrument of claim 1 wherein the sparger comprises a sparger outlet and the chemical reactor comprises a chemical reactor inlet, and further comprising a tube connected between the sparger outlet and the chemical reactor inlet, the tube forming a portion of a flow path that carries the purgeable organic carbon POC.

9. A method of measuring dissolved carbon in a sample liquid, comprising:
receiving a sample liquid, liquid water and a base liquid at valve inlets of a valve having a valve control input and a first valve outlet;
selectively pumping the liquids from the valve inlets to the first valve outlet with a syringe pump coupled to the valve, the syringe pump including a pump control input;
receiving the sample liquid and a quantity of the base liquid from the first valve outlet at a sparger;
providing purgeable organic carbon POC gasses from the sparger during a first time interval;
receiving the purgeable organic carbon POC gasses in a chemical reactor;
generating carbon dioxide in the chemical reactor during the first time interval;
coupling a control actuation sequence to the valve control input and the pump control input; and
coupling the carbon dioxide to an analyzer coupled to a controller, the controller generating a first analyzer output representing a concentration of dissolved purgeable organic carbon POC in the sample liquid.

10. The method of claim 9 further comprising:
receiving an oxidizing liquid at a valve inlet on the valve; and
receiving a quantity of the oxidizing liquid from the valve at the sparger to generate inorganic carbon IC gasses;
providing the inorganic carbon IC gasses to the reactor during a second time interval;
generating carbon dioxide in the chemical reactor during the second time interval; and
generating a second analyzer output representative of the concentration of dissolved inorganic carbon IC in the sample liquid.

11. The method of claim 10 wherein the liquids in the sparger comprise non-purgeable organic carbon NPOC and:
providing the liquids in the sparger to the chemical reactor during a third time interval,
generating carbon dioxide in the chemical reactor during the third time interval; and
generating a third analyzer output representative of the concentration of dissolved non-purgeable organic carbon NPOC in the sample liquid.

12. The method of claim 10, further comprising
providing a second valve outlet on the valve;
providing a UV reactor as the chemical reactor; and
providing a UV reactor liquid input on the UV reactor to receive a quantity of the oxidizing liquid from the second valve outlet.

13. The method of claim 12, further comprising
receiving carrier gas at a gas inlet of a gas valve having a gas valve control input;
receiving the carrier gas from the gas valve at a sparger gas inlet on the sparger; and
providing the control actuation sequence to the gas valve control input.

14. The method of claim 9, further comprising:
providing a moisture control system MCS having an MCS inlet coupled to the UV reactor outlet to receive the carbon dioxide and a MCS outlet; and
providing a scrubber having a scrubber inlet coupled to the MCS outlet and a scrubber outlet providing the carbon dioxide to the analyzer.
